(19) United States
(12) Patent Application Publication
(10) Pub. No.: US 2026/0274854 A1
(43) Pub. Date: Sep. 17, 2026

(54) METHODS OF SYNTHESIZING 14-B-AMINOMORPHANS AND SALTS THEREOF

(71) Applicant: Board of Regents, The University of Texas System, Austin, TX (US)

(72) Inventors: Charles Patrick FRANCE, Austin, TX (US); James H. WOODS, Austin, TX (US); Alexander KOLCHINSKI, Devens, MA (US); Nicholas PASCHALIDES, Devens, MA (US); Sergiy KRASUTSKY, Devens, MA (US)

(21) Appl. No.: 19/472,201

(22) PCT Filed: Apr. 4, 2024
(86) PCT No.: PCT/US2024/022940

§ 371 Date: Oct. 3, 2025

Related U.S. Application Data
(60) Provisional application No. 63/494,078, filed on Apr. 4, 2023.

(51) Int. Cl. C07D 489/06

(52) U.S. Cl. CPC C07D 489/06

(57) ABSTRACT

Disclosed herein are methods for synthesizing 14-β-amino morphinone derivatives, including methocinnamox and related compounds.

### CROSS-REFERENCE TO RELATED APPLICATION

This application claims the benefit of U.S. Provisional Application 63/494,078, filed Apr. 4, 2023, the contents of which is hereby incorporated in its entirety.

### STATEMENT OF GOVERNMENT SUPPORT

This invention was made with government support under R01DA048417 and UG3DA048387, awarded by the National Institute of Drug Abuse, National Institutes of Health.

The government has certain rights in the invention.

### FIELD OF THE INVENTION

The present invention relates method of synthesizing methocinnamox and other 14-β-amino opioid derivatives.

### BACKGROUND

Methocinnamox (“MCAM”) is long-acting and highly selective opioid receptor

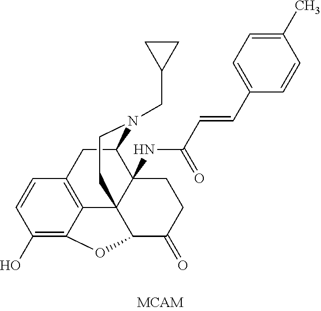 [SMILES: Cc1ccc(/C=C/C(=O)N[C@@]23CCC(=O)[C@@H]4Oc5c(O)ccc6c5[C@@]42CCN(CC2CC2)[C@@H]3C6)cc1]

Compared with other opioid receptor antagonists, MCAM binds to opioid receptors for extended periods of time, so that its effects are very long lasting compared with other antagonists. As such MCAM is an effective medication for treating opioid abuse, because of its long duration of action. A subject receiving MCAM is less likely to seek out opioids, because the MCAM effectively renders the subject immune to the euphoric effects of the opioid. MCAM is also useful for treating opioid overdose, because of its long duration of protection. Moreover, the risk of re-narcotization is much less likely when overdose is treated with MCAM than other antagonists.

There remains a need for methods of synthesizing MCAM and other opioid derivatives. There remains a need for improved pharmaceutically acceptable salts of MCAM for use in therapeutic compositions. There remains a need for improved crystalline forms of MCAM for use in therapeutic compositions.

### BRIEF DESCRIPTION OF THE FIGURES

FIG. 1 depicts an x-ray diffraction pattern of MCAM free base Form 1.

FIG. 2 depicts differential scanning calorimetry analysis of MCAM free base Form 1.

FIG. 3 depicts an x-ray diffraction pattern of MCAM free base Form 1 before and after DVS.

FIG. 4 depicts x-ray diffraction patterns of MCAM maleate salt Form 2 before and after DVS.

FIG. 5 depicts differential scanning calorimetry analysis of MCAM maleate salt Form 2.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of salts, which may be pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

The term “alkoxy” refers to an alkyl group, as defined herein, appended through an oxygen atom. In some embodiments, the alkoxy moiety has 1 to 8 carbon atoms (“C₁₋ₛ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

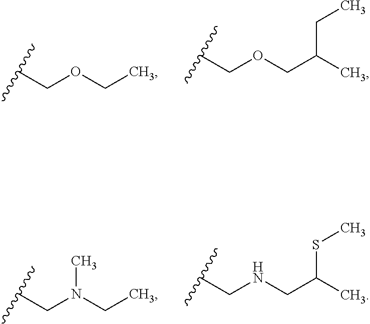

The term “heteroalkyl” preceded by a separate heteroatom refers to a heteroalkyl group bonded through the specified heteroatom. By way of example, a OC₁₋₆heteroalkyl group includes, but it not limited to, the following structures:

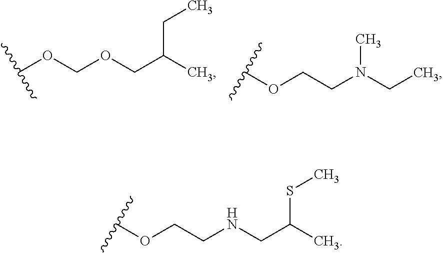

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄aryl”). In some embodiments, an aryl group has 6 ring carbon atoms (“C₆aryl”; e.g., phenyl). In some embodiments, an aryl group has 10 ring carbon atoms (“C₁₀aryl”; e.g., naphthyl such as 1-naphthyl and 2-naphthyl). In some embodiments, an aryl group has 14 ring carbon atoms (“C₁₄aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

When used in the context of a possible substituent, “D” refers to deuterium and indicates that the isotopic enrichment (relative to H and T) at the indicated position is at least 50%.

The term “halo” or “halogen” refers to fluorine (fluoro, —F), chlorine (chloro, —Cl), bromine (bromo, —Br), or iodine (iodo, —I).

The term “oxo” refers to the group ═O, and the term “thiooxo” refers to the group ═S.

The term “cyano” refers to the group —CN.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

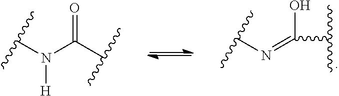

The prevalence of one tautomeric form over another will depend on the specific chemical compound as well as its local chemical environment. Unless specified to the contrary, the depiction of one tautomeric form is inclusive of all possible tautomeric forms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

The term “MCAM” refers to the compound methocinnamox.

Disclosed herein are methods for preparing compounds of Formula (1):

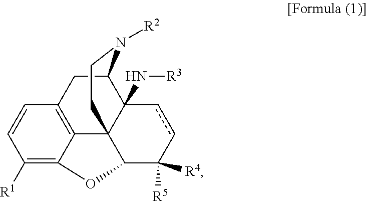

or a salt thereof. The compound of Formula (1) may be a compound of Formula (1a) or (1b):

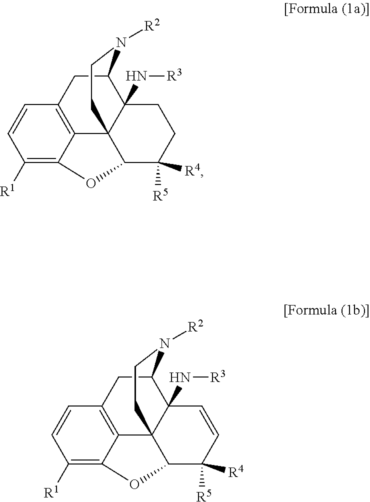

wherein
R¹ is selected from H, OH, OC₁₋₃alkyl, or OC(═O)C₁₋₃alkyl;
R² is selected from H, (CH₂)ₙR⁸, wherein n is 1-4 and R⁸ is selected from C₃₋₈cycloalkyl, and CH═CH₂;
R³ is selected from:
   (a) H; or
   (b) C(═O)X—Y—R⁷, wherein X is null, O, or NH; Y is null, C₁₋₄alkyl, C₁₋₄alkyenyl, or C₁₋₄alkynyl, and R⁷ is H, aryl, or C₁₋₈heterocyclyl; and
R⁴ and R⁵ together form an oxo, or one of R⁴ and R⁵ is OH and the other of R⁴ and R⁵ is H.

In certain implementations, the compound prepared is methocinnamox, i.e., R¹ is OH, R² is CH₂cyclopropyl, R³ is 4-methylcinnamonyl, and R⁴ and R⁵ together form an oxo.

In some implementations, the method includes the step of reacting a compound of Formula (2)

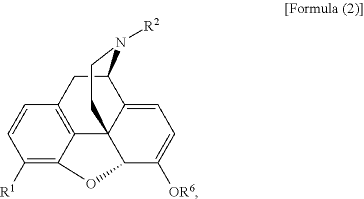

or a salt thereof, wherein R¹ and R² are as defined above, and R⁶ is selected from OC₁₋₄alkyl, OC(═O)C₁₋₄alkyl, OSi(C₁₋₄alkyl)₃;
with a compound having the formula R*—N═O, wherein R* is C(═O)X′—Y′—R^{7′}, wherein X′ is null, O, or NH; Y′ is null, C₁₋₄alkyl, C₁₋₄alkyenyl, or C₁₋₄alkynyl, and R^{7′} is H, aryl, or C₁-8heterocyclyl;
to form a cycloaddition adduct (a cycloadduct) having the formula:

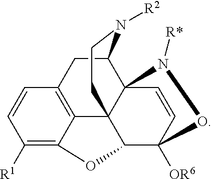

or a salt thereof. The cycloadduct may be reduced to give a compound of Formula (3a) or (3b):

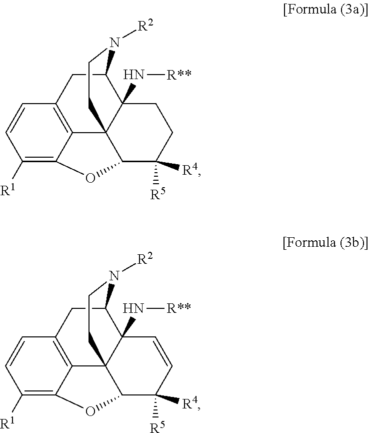

or a salt thereof, wherein R** is H or R*, and R¹, R², R⁴, R⁵, and R⁶ are as defined above.

In some implementations the compound of Formula (3a) is produced in excess of Formula (3b). In certain implementation the compound of Formula (3a) is produced in at least 80 mol %, at least 90 mol %, at least 95 mol %, at least 97.5 mol %, or 99 mol % relative to the total mole amount of Formula (3a)+Formula (3b). In other implementations the compound of Formula (3b) is produced in excess of Formula (3a). In certain implementation the compound of Formula (3b) is produced in at least 80 mol %, at least 90 mol %, at least 95 mol %, at least 97.5 mol %, or 99 mol % relative to the total mole amount of Formula (3a)+Formula (3b).

In some implementations, the cycloadduct is isolated and/or purified prior to reduction. As used herein, a compound is in isolated form if it exists in substantially pure form, separate from any other compounds, reagents, or solvents. A compound is purified by removing one or more solvents, reagents, or reaction by-products, but does not necessary denote the compound is in isolated form. For example, a compound in solution may be purified by subjecting the solution to a filtration step, but the compound itself not separated from the solvent and/or other soluble components. In some embodiments, the cycloadduct can be subject to chromatography, precipitation (which may be a crystallization), drying under vacuum or reduced pressure, solvent-solvent extraction, azeotropic distillation, or a combination thereof.

In some implementations, the cycloadduct is directly contacted with the reductant prior to any isolation or purification step. Such a process may be designated a sequential one-pot process.

In some implementations, the compound having the formula R*—N═O can be generated by:
(a) oxidizing a compound having the formula R*—NH—OH, R*—NH₂, or a combination thereof; or
(b) heating a sacrificial adduct having the formula R¹N(R*)—OR², wherein R¹ and R² together form a strained ring system.

In some implementations, the sacrificial adduct is a 9,10-dimethylanthracenyl adduct, e.g.:

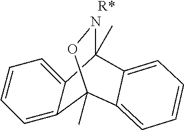

In some implementations, the compound having the formula R*—N═O is generated by combining a compound having the formula R*—NH—R^{∘}, wherein R^{∘} is H or OH, i.e., one or more of R*—NH—OH or R*—NH₂ with an oxidizing system. Exemplary oxidizing systems include one or more oxidants, for example periodate salts, hydrogen peroxide, peroxyacids, Fe(III) salts, MnO₂, and combinations thereof. In certain implementations the oxidant includes NaIO₄, BnMe₃NIO₄, or Et₄NIO₄.

In certain implementations R* is a protective group that is cleavable under reductive conditions. In some implementations, R* is benzyl (—CH₂-phenyl) or benzyloxycarbonyl —C(═O)OCH₂phenyl. In some implementations R* is a substituted benzyl or substituted benzyloxycaronyl. Exemplary substituents include para-alkyl, para-methoxy, para-nitro, para-halo and the like. In other implementation R* is 4-methylcinnaomyl, i.e.:

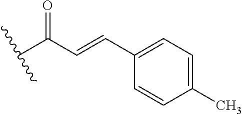 [SMILES: *C(=O)/C=C/c1ccc(C)cc1]

In some implementations, the cinnamoyl group maybe be unsubstituted or differently substituted than 4-methyl, e.g., 4-fluoro, 4-chloro, 4-bromo, 4-iodo, or 4-nitro.

In certain implementations, the compound having the formula R*—NH—OH or R*—NH₂ is oxidized separately from the compound Formula (2), and then combined with the compound of Formula (2) as the compound R*—N═O. In other implementations, the compound having the formula R*—NH—OH or R*—NH₂ is oxidized in the presence of the compound Formula (2). In exemplary embodiments, the compound of Formula (2) and compound having the formula R*—NH—OH or R*—NH₂ can be combined in a reaction mixture, to which an oxidant can then be added (or the mixture of the compound of Formula (2) and compound having the formula R*—NH—OH or R*—NH₂ can be added to an oxidant). In other implementation, the compound of Formula (2) and oxidant can be combined in a reaction mixture, which is then combined with a compound having the formula R*—NH—OH or R*—NH₂.

In certain embodiments, the reaction mixture can include a solvent, for instance an aqueous solvent, organic solvent, or a combination thereof. In certain implementations, a mixture of aqueous solvent and organic solvent can be used. In some implementations the organic solvent can include ethyl acetate (EtOAc), isopropyl acetate (IPAc), butyl acetate, methyl ethyl ketone, methyl butyl ketone, methyl isobutyl ketone, n-butanol, ethyl benzene, toluene, xylene, pentane, n-hexane, n-heptane, trichloroethane, 1,1-dichloroethane, 2,3-dichloroethane, dichloromethane (DCM), and combinations thereof. In certain implementations the organic solvent can include ethyl acetate.

In some implementations, the compound of Formula (2) may be provided in the form of an acid addition salt, while in other implementations the compound of Formula (2) may be provided as the free base. In certain implementations, the compound of Formula (2) as the free base is combined with a periodate salt in a solvent, for instance a mixture of aqueous and organic solvents. In some implementation the periodate salt is one or more of NaIO₄, BnMe₃NIO₄, or Et₄NIO₄. In certain implementations the oxidant can be present in an amount (relative to the compound of Formula (2)) from 1-1.5 equivalents, from 1-1.25 equivalents, from 1-1.1 equivalents, from 1.05-1.15 equivalents, from 1.1-1.25 equivalents, from 1.1-1.3 equivalents, or from 1.25-1.5 equivalents.

In certain implementations, the oxidant (optionally in combination with the compound of Formula (2)) is in solution at a temperature from −10° C. to 25° C., from −10° C. to 0° C., from −5° C. to 5° C., from 0° C.-10° C., from 0° C. to 25° C., from 5° C. to 20° C., from 5° C. to 15° C., or from 10° C. to 20° C. To this mixture the compound having the formula R*—NH—OH or R*—NH₂ is added. The compound of the formula R*—NH—OH or R*—NH₂ may be added as a solution, preferably dissolved in the same organic solvent present in the reaction mixture. In certain implementations, the compound of Formula (2) and the compound formula R*—NH—OH or R*—NH₂ are in a solution at a temperature from −10° C. to 25° C., from −10° C. to 0° C., from −5° C. to 5° C., from 0° C.-10° C., from 0° C. to 25° C., from 5° C. to 20° C., from 5° C. to 15° C., or from 10° C. to 20° C., and to this solution the oxidant is added.

The reaction may be monitored by assessing the consumption of the compound of Formula (2), for instance by HPLC, NMR, mass spectroscopy, and the like. In certain embodiments, at least 90% of the compound of the compound of Formula (2) is converted to the cycloadduct. In some implementations, at least 92%, at least 94%, at least 96%, at least 98%, or at least 99% of the compound of Formula (2) is converted to the cycloadduct.

Upon completion of the cycloaddition reaction, the cycloadduct may be purified and/or isolated if desired. In some implementations, the reaction mixture may be filtered to remove precipitates, and in some implementations the filtrate extracted with organic solvent to separate the cycloadduct. The cycloadduct may be further purified using chromatography or crystallization.

In other embodiments, the cycloadduct is not purified or isolated. In such embodiments excess oxidant may be destroyed with a mild reductant, for example with methionine, thiosulfate salts or sulfite salts. The reaction mixture may then be combined with a reduction system as described herein to reduce the N—O bond.

In some implementations the cycloadduct is reduced using one or more of:
(a) a metal in combination with a hydrogen source;
(b) a metal hydride; or
(c) a metal in combination with an acid.

In certain implementations the hydrogen source is H₂, 1,4-cyclohexadiene, ammonium formate, sodium dithionite, or a combination thereof. Exemplary metals include palladium, platinum, rhodium, ruthenium, nickel, copper, iron, tin, zinc, or a combination thereof. In some implementations the metal is a homogenous catalyst, while in some implementations the metal is a heterogenous catalyst. In some implementations the metal is palladium or platinum, for instance of a solid support like carbon or carbonate salt. In certain implementations the metal is Pd/C, Pd/CaCO₃, or Pd/BaCO₃. In some implementations the reduction system includes Raney nickel, LiAlH₄, NaBH₄, NaBH₃CN, diisobutylaluminum hydride (DIBAL), Zn/acid, Fe/acid, or a combination thereof.

In certain implementations the cycloadduct is reduced under catalytic hydrogenation conditions using hydrogen gas in the presence of a catalyst like Pd, Pd/C, Pd/CaCO₃, PtO₂, Pt/C, RhCl(PPH₃)₃, Rh₂Cl₂(cod)₂, Crabtree's catalyst, Nishimura's catalyst, or a combination thereof.

In certain implementations, the cycloadduct may be combined with alcohol such as methanol (MeOH), isopropanol, ethanol, n-propanol, or a combination thereof. In some implementations, an acid may be present. Exemplary acids include mineral acids like hydrochloric acid, sulfuric acid, nitric acid, phosphoric acid, and the like. In some embodiments the acid is an organic acid like formic acid or acetic acid. The pH of the resulting mixture should be between 2-6, 2-5, between 2-4, between 2-3, between 3-4, between 3-5, or between 4-6. When an organic acid is used, it can be present in an amount (relative to the total solvent volume) of 1-20%, 1-10%, 5-10%, 5-15%, or 10-20%.

The reduction reaction may be monitored by assessing the consumption of the cycloadduct, for instance by HPLC, NMR, mass spectroscopy, and the like. In certain embodiments, at least 90% of the cycloadduct is consumed. In some implementations, at least 92%, at least 94%, at least 96%, at least 98%, or at least 99% of the cycloadduct is consumed.

In certain implementation R* is chosen such that the reduction process produces a compound of Formula (4):

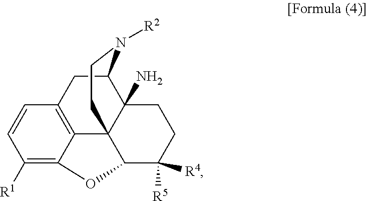

or a salt thereof, wherein R¹, R², R⁴ and R⁵ are as defined above. In some implementations, R² is CH₂cyclopropyl, R⁴ and R⁵ together form an oxo, and R¹ is OH or CH₃.

In some the reduction product is the compound of Formula (4a):

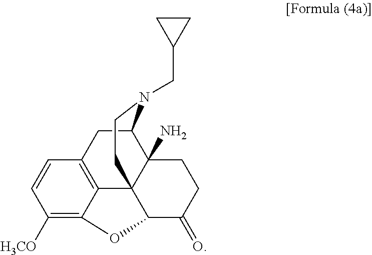 [SMILES: COc1ccc2c3c1O[C@H]1C(=O)CC[C@@]4(N)[C@@H](C2)N(CC2CC2)CC[C@]314]

In certain implementations, the reduction process is selective for the compound of Formula (4a) (MW=354.45), and does not produce an over-reduced product, e.g., compound having MW=356.45. The presence of the over-reduced product may be assessed using HPLC-MS and related techniques. In certain embodiments, no more than 10 mol %, relative to the compound of Formula (4a) of the over-reduced product is formed. In some implementations, no more than 5 mol %, no more than 2.5 mol %, no more than 1.0 mol %, or no more than 0.5 mol % over the over-reduced product (relative to the compound of Formula (4a)) is produced.

The compound of Formula (4) may be isolated and/or purified prior to further transformations, or it may be directly acylated as described below. In certain embodiments, the compound of Formula (4) is filtered to remove any heterogenous hydrogenation catalyst. The filtrate may be directly used in the acylation reaction, or it may be further purified, for example to remove water and residual acid, by solvent-solvent extraction with an aqueous base.

In some implementations the compound of Formula (4) or (4a) may be reacted with a compound of Formula (Z):

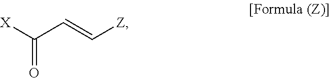

wherein X is halo, C₁₋₆alkoxy, C₆₋₁₀aryloxy, or C₁₋₆heteroaryl, each optionally substituted one or more times by a substituent in each case independently selected from halo, nitro, cyano, C₁₋₆ alkyl, or C₁₋₆alkoxy, and Z is optionally substituted aryl or heterocyclyl.

In some implementations Z has the formula:

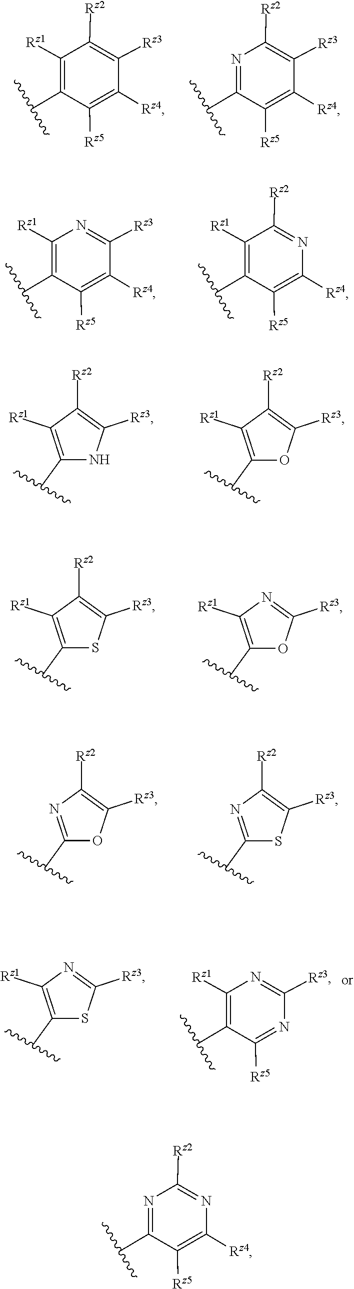

wherein:
R^{z1} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z2} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z3} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z4} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z5} is H, D. OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
wherein any two adjacent R^{z} groups may together form a fused aryl or heterocyclyl ring.

In some implementations the compound of Formula (4) or (4a) may be reacted with a compound of Formula (X):

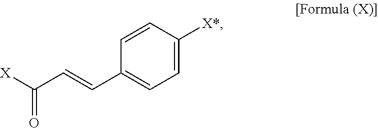

wherein X is halo, C₁₋₆alkoxy, C₆₋₁₀aryloxy, or C₁₋₆heteroaryl, each optionally substituted one or more times by a substituent in each case independently selected from halo, nitro, cyano, C₁₋₆alkyl, or C₁₋₆alkoxy;
and X* is H, C₁₋₄alkyl, halo (i.e., F, Cl, Br, or I), or nitro,
to give a compound of Formula (5):

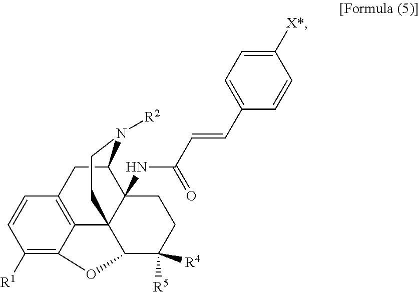

or a salt thereof. In certain implementations, X is halo, preferably Cl, N-imidazolyl, phenoxy or substituted phenoxy, e.g., 4-nitrophenoxy, 4-chlorophenoxy, 4-fluorophenoxy, pentafluorophenoxy, 4-cyanophenoxy. In some implementations, X* is CH₃.

In some implementations, the compound of Formula (x) is used in stoichiometric excess relative to the compound of Formula (4) or (4a). In certain implementations, the compound of Formula (x) can be present in an amount (relative to the compound of Formula (4) or (4a)) from 1-1.5 equivalents, from 1-1.25 equivalents, from 1-1.1 equivalents, from 1.05-1.15 equivalents, from 1.1-1.25 equivalents, from 1.1-1.3 equivalents, or from 1.25-1.5 equivalents.

The acylation reaction may be carried out in an organic solvent, for example ethyl acetate, isopropyl acetate, butyl acetate, methyl ethyl ketone, methyl butyl ketone, methyl isobutyl ketone, ethyl benzene, toluene, xylene, pentane, n-hexane, n-heptane, trichloroethane, 1,1-dichloroethane, 2,3-dichloroethane, dichloromethane, and combinations thereof, in the presence of a base, for example an amine base such as triethylamine, pyridine, diethyl isopropylamine, tributylamine, 2,6-lutidine, pempidine, DABCO, DBU, DBN, and the like. In certain implementations, the amine base is used in stoichiometric excess relative to the compound of Formula (4) or (4a). In certain implementations, the amine base can be present in an amount (relative to the compound of Formula (4) or (4a)) from 1-1.5 equivalents, from 1-1.25 equivalents, from 1-1.1 equivalents, from 1.05-1.15 equivalents, from 1.1-1.25 equivalents, from 1.1-1.3 equivalents, or from 1.25-1.5 equivalents. In some implementations, the mole ratio of amine base to compound of Formula (X) is about 1:1.

The acylation reaction may be conducted in the presence of a catalyst, for example dimethylaminopyridine.

In some embodiments, the acylation may be conducted using the free cinnamyl acid, i.e., X is OH.

In certain implementations the compound of Formula (5) is characterized when R¹ is OCH₃ i.e., a compound of Formula (5) —OCH₃. In further implementations the R¹ group may be converted from methoxy to hydroxy [a compound of Formula (5) —OH] by a demethylation reaction. Exemplary demethylating agents include BBr₃, LiPPH₂, L-selectride, HBr, 2-(diethylamino)ethanethiol, or a combination thereof. In certain implementations the demethylating agent is BBr₃. In certain implementations, the BBr₃ can be present in an amount (relative to the compound of Formula (5)) from 2-10 equivalents, from 2-5 equivalents, from 5-10 equivalents, from 3-5 equivalents, from 2-4 equivalents, or from 1-3 equivalents.

In certain embodiments, the compound of Formula (5) —OCH₃ is added as an organic solution to a vessel containing an organic solution of the demethylating agent. In some implementations the solvent used to dissolve the compound of Formula (5) —OCH₃ is the same as the solvent used to dissolve the demethylating agent. Exemplary solvents include ethyl acetate, isopropyl acetate, butyl acetate, methyl ethyl ketone, methyl butyl ketone, methyl isobutyl ketone, ethyl benzene, toluene, xylene, pentane, n-hexane, n-heptane, trichloroethane, 1,1-dichloroethane, 2,3-dichloroethane, dichloromethane, and combinations thereof. In certain implementations, the solvent is dichloromethane and the demethylating agent is BBr₃.

The mixture may be maintained at a temperature from −10° C. to 25° C., from −10° C. to 0° C., from −5° C. to 5° C., from 0° C.-10° C., from 0° C. to 25° C., from 5° C. to 20° C., from 5° C. to 15° C., or from 10° C. to 20° C.

In certain embodiments, at least 90% of the compound of Formula (5) —OCH₃ is converted to the compound of Formula (5) —OH. In some implementations, at least 92%, at least 94%, at least 96%, at least 98%, or at least 99% of the compound of Formula (5) —OCH₃ is converted to the compound of Formula (5) —OH. The reaction progress may be monitored using HPLC, NMR, mass spectrometry, and the like. Upon completion of the demethylation reaction, excess demethylating agent may be quenched and the compound of Formula (5) —OH may be purified by solvent-solvent extraction into a polar non-protic solvent such as ethyl acetate, isopropyl acetate, butyl acetate, methyl ethyl ketone, methyl butyl ketone, methyl isobutyl ketone, trichloroethane, 1,1-dichloroethane, 2,3-dichloroethane, dichloromethane, and combinations thereof. In some implementations, the product is precipitated by addition of water or an C₁₋₄alkyl alcohol, for example methanol, ethanol, or isopropanol. The resulting product can be isolated by filtration and rinsing the precipitate with water or C₁₋₄alkyl alcohol. In some implementations, the purity (by HPLC) of the demethylated product is at least 90%, at least 95%, at least 97.5%, at least 99%, or at least 99.5%.

Also disclosed herein are methods of installing a cyclopropyl group into a morphinan derivative. In some implementations, a compound of Formula (2a):

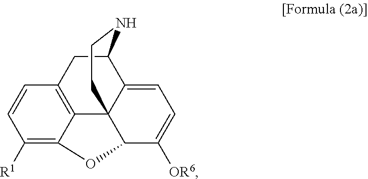

or salt thereof, wherein R¹ and R⁶ are as defined above, is combined with cyclopropane carboxaldehyde or functional equivalent thereof, under reducing conditions to give a compound of Formula (2a-cy):

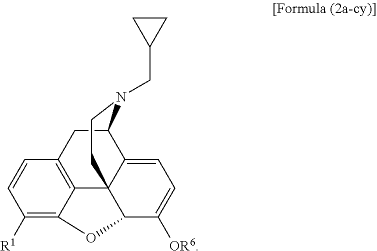

In some implementations, the functional equivalent of cyclopropane carboxaldehyde is a cyclopropane carboxaldehyde dialkyl acetal having the formula:

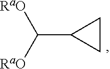

wherein R^{a} is a C₁₋₄alkyl group, or the two R^{a} groups together form a five or six membered ring. Exemplary acetals include dimethyl acetal, diethyl acetal, dipropyl acetal, ethylene acetal, and propylene acetal. In some implementations, the compound of Formula (2a-cy) is combined with the cyclopropyl carboxaldehyde or derivative thereof in an organic solvent, and the solution is then added to a reaction vessel containing the reductant and solvent. Exemplary solvents include ethyl acetate, isopropyl acetate, butyl acetate, methyl ethyl ketone, methyl butyl ketone, methyl isobutyl ketone, ethyl benzene, toluene, xylene, pentane, n-hexane, n-heptane, trichloroethane, 1,1-dichloroethane, 2,3-dichloroethane, dichloromethane, and combinations thereof. In some implementations, the organic solvent is dichloromethane. The solution may further include a base, for example an amine base such as triethylamine, pyridine, diethyl isopropylamine, tributylamine, 2,6-lutidine, pempidine, DABCO, DBU, DBN, and the like. In some implementations, the amine base is present in an amount of at least 1.5 equivalents relative the compound of Formula (2a). In certain implementations, the cyclopropane carboxaldehyde or functional equivalent thereof can be present in an amount (relative to the compound of Formula (2A)) from 1-1.5 equivalents, from 1-1.25 equivalents, from 1-1.1 equivalents, from 1.05-1.15 equivalents, from 1.1-1.25 equivalents, from 1.1-1.3 equivalents, or from 1.25-1.5 equivalents.

In certain implementations, the reductant is a borohydride, lithium hydride, or aluminum hydride. In some implementations, the reductant is sodium borohydride, sodium cyanoborohydride, sodium triacetoxyborohydride (STAB-H), or a combination thereof. In certain implementations, the reductant can be present in an amount (relative to the compound of Formula (2a)) from 1-2 equivalents, from 1-1.5 equivalents, from 1.5-2 equivalents, from 1.25-1.75 equivalents, from 1.5-3 equivalents, or from 2-4 equivalents.

The reductant solution may be maintained at a temperature from −10° C. to 25° C., from −10° C. to 0° C., from −5° C. to 5° C., from 0° C.-10° C., from 0° C. to 25° C., from 5° C. to 20° C., from 5° C. to 15° C., or from 10° C. to 20° C. In some implementations, the compound of Formula (2a) and cyclopropyl carboxaldehyde (or derivative thereof), is added to reductant at a temperature from 0° C.-15° C. Upon completion of the addition, the reaction mixture may be allowed to warm to room temperature and monitored for the consumption of the compound of Formula (2a). In some implementations, at least 92%, at least 94%, at least 96%, at least 98%, or at least 99% of the compound of Formula (2a) is converted to the compound of Formula (2a-cy).

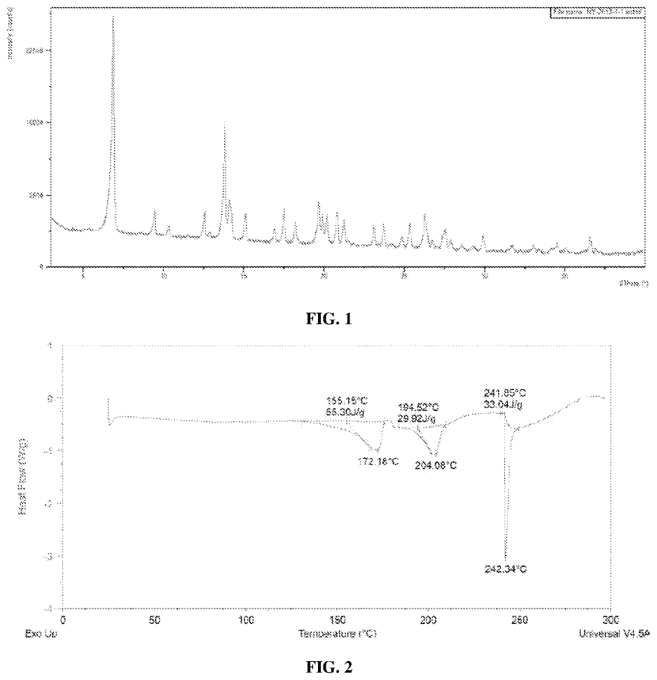

Also disclosed herein are novel salts and polymorphs of MCAM. In some implementations, MCAM as provided as a crystalline free base. In some implementations MCAM is provided as crystalline free base Form 1. Free base Form 1 can characterized by a DSC exotherm at 208.8° C. (27.3 J/g) and a DSC endotherm at 261.2° C. (71.4 J/g). In some implementations MCAM free base Form 1 is provided as plate shape crystals, having widths from 5-20 μm. MCAM free base Form 1 produces an x-ray diffraction pattern as shown in FIG. 1.

In some implementations, MCAM is provided as a maleate salt, for example a crystalline maleate salt (1:1). In some implementations MCAM can be provided as maleate salt Form 2. Maleate salt Form 2 can be characterized by DSC endotherms at 155.2° C., 194.5° C., and 241.9° C. (FIG. 2). Maleate salt Form 2 is a monohydrate and can be characterized by a water content of about 3% by KF, the theoretical content for monohydrate being 2.9%. Maleate salt form 2 is stable, undergoing no polymorphic changes when stored at 40° C./75% RH for 7 days.

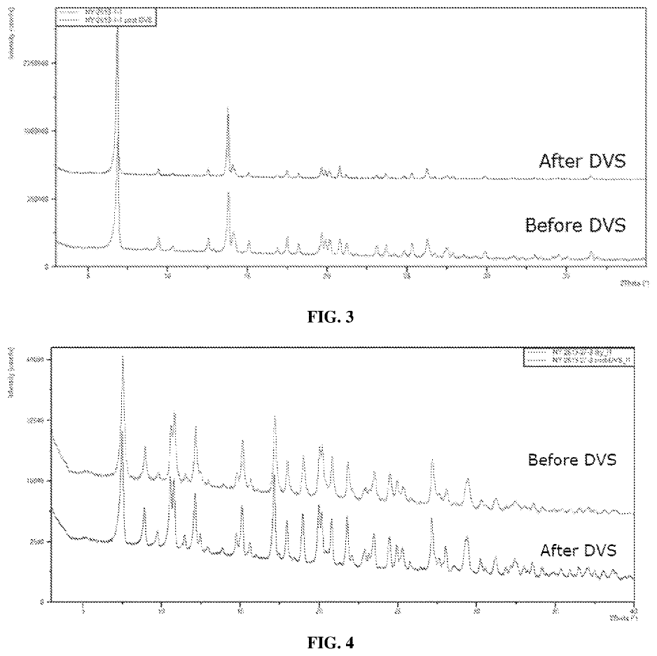

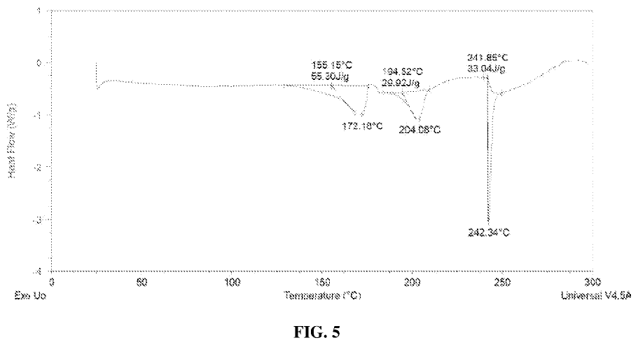

MCAM maleate salt Form 2 can be characterized by x-ray diffraction peaks (°2Θ) of 7.9±0.2, 13.7±0.2, and 16.3±0.2. MCAM maleate salt Form 2 can be further characterized by peaks at 9.8±0.2, 10.2±0.2, 15.1±0.2, 19.4±0.2, 21.4±0.2, and 22.8±0.2. MCAM maleate salt Form 2 can be further characterized by peaks at 7.1±0.2, 9.0±0.2, 12.3±0.2, and 13.4±0.2. MCAM maleate salt Form 2 can be further characterized by peaks at 11.8±0.2, 12.7±0.2, 14.2±0.2, 21.7±0.2, and 24.5±0.2. MCAM maleate salt Form 2 can be further characterized by having 5 or more peaks selected from 14.5±0.2, 16.8±0.2, 17.4±0.2, 18.1, ±0.2, 18.6±0.2, 20.2±0.2, 22.0 0.2, 22.4±0.2, 23.5±0.2, 24.9±0.2, 25.4±0.2, and 26.4±0.2. MCAM maleate salt Form 2 produces an x-ray diffraction pattern as shown in FIG. 3 (before and after dynamic vapor sorption (DVS)).

In some implementations, MCAM is provided as the maleate salt Form 3. Form 3 is anhydrous. MCAM maleate salt Form 3 can be obtained when Form 2 is heated to 40° C. under vacuum for at least 24 hours.

### EXAMPLES

The following examples are for the purpose of illustration of the invention only and are not intended to limit the scope of the present invention in any manner whatsoever.

### Example 1: Preparation of Northebaine

Thebaine (1 eq, potency typically 85% by Q-NMR) and MeCN (6.5 vol) were added to a jacketed reactor, equipped with overhead stirrer, temperature probe, distillation set-up, and N₂ inlet charge at 20° C. (20±5° C.). An additional MeCN (2 vol) was charged and the mixture was concentrated to 6.5 vol via atmospheric distillation and analyzed for water content by KF. This step was repeated until water content by KF<0.1 wt %. The temperature of the reaction mixture to adjusted to 55° C., diisopropyl azodicarboxylate (DIAD) (1.1 eq.) was added slowly over 40-50 mins. The resulting slurry was heated to 78° C. (78±2° C.) with stirring under nitrogen for 6 h.

Upon consumption of the thebaine (IPC-HPLC≤2.0% AUC thebaine), the reaction mixture was transferred to a different reactor containing Py·HCl (2.0 eq.) and MeCN (1 vol) (pre-heated to 60° C.) over 1.5 hours. Water (14 wt. %) was added and the reaction mixture was warmed to 78° C. (78±2° C.) with stirring under nitrogen atmosphere overnight (16 h). The reaction was cooled to 30° C. over 2 h, and the resulting slurry was filtered and washed with MeCN (2×2 vol). The wet filter-cake was dried in a vacuum overnight at 35° C. to obtain northebaine HCl as a white powder. Typical yield 61.47%, typical purity 98.91% AUC by HPLC.

### Example 2: Preparation of N-cyclopropylmethylnorthebaine

Northebaine HCl (1 eq.) and DCM (10 vol.) were added to a jacketed reactor at 20±5° C. Triethylamine (2 eq.) was added over 20-30 mins keeping reaction temperature at 20±5° C. Cyclopropane carboxaldehyde (1.1 eq.) was added over 10 min, and the resulting mixture is cooled to 10° C. This solution was added to sodium triacetoxy borohydride STAB-H (1.5 eq.) in DCM (5 vol) at 10±5° C. over 30 min. After completion of the transfer, the mixture was warmed to 20±2° C. and stirred overnight. The reaction mixture was added to a quench solution (aqueous 10 wt. % NaOH containing 5 wt. % NaCl (10 vol)) at 10±5° C. over 30 min. The mixture was warmed to 20±5° C. with stirring for 5 min at 20±5° C., and the organic layer was separated. The aqueous layer was extracted twice with DCM (2×10 vol.), and the combined organics were concentrated to 5 vol. IPAc (5 vol) was added and the resulting mixture was concentrated by atmospheric distillation. The IPAc chase was repeated until DCM concentration in IPAc was <1.0 wt %. In practice 2 IPAc chases were sufficient to reduce the DCM concentration to desirable level. The resulting solution was filtered and diluted with IPAc (5 vol). The solution was warmed 60° C. with stirring under nitrogen, and HCl in ethanol (2.5 M, 2 eq) was added. The resulting slurry was cooled to 20±2° C. over 2 h. The white solids were filtered, washed with IPAc, and dried in a vacuum oven at 35° C. Typical yield 85%, typical purity 99.2% AUC by HPLC.

### Example 3: Formation of Cycloadduct

The HCl salt of example 2 (1 eq.) was dissolved in EtOAc (4 vol) and combined with 1 N. aq. NaOH. The organic phase was separated and the aqueous phase extracted with EtOAc (4 vol). The combined organics were washed with water (pH 7-8). The organic solution was combined with water (6 vol) and the biphasic mixture was cooled to 10° C. with sufficient agitation. NaIO₄ (1.2 eq) was added with stirring at 10° C. A solution CBz-NHOH (1.1) in EtOAc (4 vol) was added to the reaction mixture over 50-60 min at 10±5° C. Upon consumption of the diene (>99% conversion), the reaction mixture was filtered and filtrate was washed with EtOAc (2 vol). The organic phase was separated, and aqueous phase washed with EtOAc (4 vol). The combined organics were concentrated to give an amber foam, which was purified by silica gel chromatography using 3:1 heptane:EtOAc, then 2:1 heptane:EtOAc. Typical purity 95% by HPLC, typical yield 88%.

### Example 4: Cycloadduct Reduction

The cycloadduct (1 eq.) was dissolved in methanol (10 vol) and transferred to a 2 L Parr reactor. A solution of MeOH (10 vol) and AcOH (2 vol) was used to transfer residual cycloadduct and Pd/CaCO₃ (20 wt %) to the reactor (keep fill volume around 66%). The mixture was hydrogenated at 50 psig hydrogen pressure and 25° C. (rpm 370-380). The reaction may be monitored for consumption of starting material and over-reduced product by IPC-HPLC. If conversion not complete, the catalyst may be refreshed. Pre-washed celite (1 wt) was added, and the mixture is filtered through Celite (2 wt. washed with 4 vol MeOH). The filter cake was washed with MeOH (10 vol) and the combined organics evaporated. The residue was diluted in DCM (10 vol) and washed with 3 N NaOH (10 vol) and then brine (10 vol). The resulting organics were concentrated to 5 vol., and then co-evaporated with DCM until water content <0.01 wt %) (KF).

The free amine (1 eq) was combined with DCM (5 vol) and triethylamine (1.1 eq). 4-Methyl-cinnamoyl chloride (1.1 eq.) was added as a solution in approximately 5 vol toluene by weight over 30 min. The reaction mixture was stirred for 6 hr. (target >99% conversion). The reaction was quenched by slow addition of aqueous 5 wt % NaHCO₃ (10 vol) solution containing 5 wt % NaCl keeping temperature below 30° C. and stirred for 10 min. The organic layer was separated and the aqueous layer was extracted once with DCM (10 vol). The organics were concentrated to give a foam, which was purified by silica get chromatography using a gradient elution of 10%, 20%, and 30%, and 40% ethyl acetate in heptane. Typical purity 96% by HPLC, typical yield 50%.

### Example 5: Demethylation

The product of Example 4 (1 eq.) was dissolved in DCM (10 vol), and slowly added to a solution of BBr₃ in DCM (1 M, 4 eq.) diluted with DCM (5 vol) at 5±5° C. The reaction mixture was stirred at 5±5° C. for 2 h (target >99% conversion). The reaction mixture was transferred to 50% aq. NH₄OH (20 vol) over 40 min, keeping temperature of batch at <20° C. The mixture was warmed to room temperature, and MeOH was added (7 vol). The organic layer was separated, and the aqueous phase was extracted twice with 20% MeOH in DCM (10 vol). The combined organics were concentrated, keeping the batch temperature at 40° C. MeOH (10 vol) was added, the mixture was concentrated via vacuum distillation to 10 vol. The MeOH chase was repeated and the mixture cooled to 20±5° C. The product was filtered and washed with MeOH, and then dried to give methocinnamox as a white, fine crystalline solid. Typical purity 99.7% pure by HPLC, typical yield 63% yield).

### Example 6: MCAM Maleate Salt Form 2

MCAM free base (1.005 g, 2.07 mmol) was added to 40 mL scintillation vial with 10 mL acetonitrile. The vial was capped at heated to 81° C. resulting in slurry. Maleic acid (265 mg, 2.28 mmol dissolved in 2.65 ml water) was added quickly via syringe and the slurry dissolved. The solvents were evaporated under vacuum until 5 ml remained and ~5 mL water was then added. The solvents were evaporated under vacuum to reduce the volume to ~5 mL and ~5 mL water was then added. The heat source was removed and the mixture allowed to reach room temperature over about 1.5 hours. The resulting solids were filtered, washed with water, and dried overnight at 45° C. at 29.5 mm Hg. MCAM maleate salt Form 2 was obtained (1.124 g) as a flowable, non-static white powder.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method for preparing a compound of Formula (1): 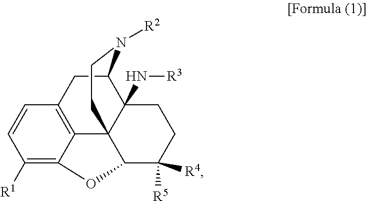
or a salt thereof, wherein
comprising combining a compound of Formula (2): 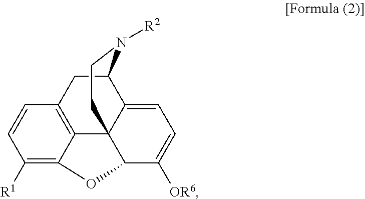
or a salt thereof, with a compound having the formula R*—N═O,
to form a cycloadduct;
combining the cycloadduct with a reduction system,
wherein
R¹ is selected from H, OH, OC₁₋₃alkyl, or OC(═O)C₁₋₃alkyl;
R² is selected from H, (CH₂)ₙR⁸, wherein n is 1-4 and R⁸ is selected from C₃₋₈cycloalkyl, and CH═CH₂;
R³ is selected from H or C(═O)X—Y—R⁷, wherein X is null, 0, or NH; Y is null, C₁₋₄alkyl, C₁₋₄alkyenyl, or C₁₋₄alkynyl, and R⁷ is H, C₁₋₄alkyl, C₁₋₄alkyenyl, aryl, or C₁₋₈heterocyclyl;
R⁴ and R⁵ together form an oxo, or one of R⁴ and R⁵ is OH and the other of R⁴ and R⁵ is H;
R⁶ is selected from OC₁₋₄alkyl, OC(═O)C₁₋₄alkyl, OSi(C₁₋₄alkyl)₃; and
R* is C(═O)X′—Y′—R^{7′}, wherein X′ is null, 0, or NH; Y′ is null, C₁₋₄alkyl, C₁₋₄alkyenyl, or C₁₋₄alkynyl, and R⁷ is H, C₁₋₄alkyl, C₁₋₄alkyenyl, aryl, or C₁₋₈heterocyclyl.

2. The method according to claim 1, wherein R² is CH₂cylopropyl, and the compound of Formula (2) is prepared by reactive a compound of Formula (2a): 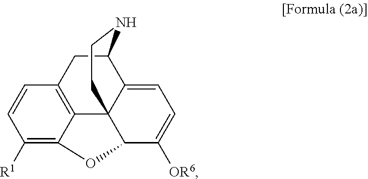
with cyclopropane carboxaldehyde, cyclopropane carboxaldehyde dialkyl acetal, or a combination thereof in the presence of a reductant.

3. The method according to claim 2, wherein the cyclopropane carboxyaldehyde dialkyl acetal comprises cyclopropane carboxyaldehyde dimethyl acetal, cyclopropane carboxyaldehyde diethyl acetal, cyclopropane carboxyaldehyde dipropyl acetal, cyclopropane carboxyaldehyde ethylene acetal, cyclopropane carboxyaldehyde propylene acetal, or a combination thereof.

4. The method according to claim 2, wherein the reductant comprises a borohydride, lithium hydride, or aluminum hydride.

5. The method according to claim 2, wherein the reductant comprises sodium borohydride, sodium cyanoborohydride, sodium triacetoxyborohydride, or a combination thereof.

6. The method according to any of claims 1-5, wherein the compound having the formula R*—N═O is generated by:
(c) oxidizing a compound having the formula R*—NH—R^{∘}, wherein R^{∘} is H or OH,
(d) heating a sacrificial adduct having the formula R¹N(R*)—OR², wherein R¹ and R² together form a strained ring system.

7. The method according to claim 6, wherein the compound having the formula R*—N═O is generated by combining a compound having the formula R*—NH—R^{∘} with an oxidizing system.

8. The method according to claim 7, wherein the oxidizing system comprises a periodate salt, hydrogen peroxide, a peroxyacid, an Fe(III) salt, MnO₂, or a combination thereof.

9. The method according to claim 8, wherein the oxidizing system comprises NaIO₄, BnMe₃NIO₄, or Et₄NIO₄.

10. The method according to claim 6, wherein the sacrificial adduct is a 9,10-dimethylanthracenyl adduct.

11. The method according to claim 6, wherein the compound having the formula R*—NH—R^{∘} is oxidized in the presence of the compound of Formula (2).

12. The method according to claim 6, wherein the compound having the formula R*—NH—R^{∘} is oxidized separately from the compound of Formula (2).

13. The method according to claim 6, wherein the compound having the formula R*—N═O is purified prior to combining with the compound of Formula (2).

14. The method according to any of claim 6, wherein the reduction system comprises one or more of:
(a) a metal in combination with a hydrogen source;
(b) a metal hydride; or
(c) a metal in combination with an acid.

15. The method according to claim 14, wherein the reduction system comprises a metal in combination with H₂, 1,4-cyclohexadiene, ammonium formate, sodium dithionite, or a combination thereof.

16. The method according to claim 15, wherein the reduction system comprises a metal selected from palladium, platinum, rhodium, ruthenium, nickel, copper, iron, tin, zinc, or a combination thereof.

17. The method according to claim 14, wherein the reduction system comprises Raney nickel, LiAlH₄, NaBH₄, NaBH₃CN, diisobutylaluminum hydride, Zn/acid, Fe/acid, or a combination thereof.

18. The method according to claim 14, wherein the reduction system comprises H₂ in combination with Pd, Pd/C, Pd/CaCO₃, PtO₂, Pt/C, RhCl(PPH₃)₃, Rh₂Cl₂(cod)₂, Crabtree's catalyst, Nishimura's catalyst, or a combination thereof.

19. The method according to any of claims 1-18, wherein R* is benzyl or benzyloxycarbonyl.

20. The method according to any of claims 1-18, wherein R³ is H.

21. The method according to any of claims 1-18, wherein R₁ is OH or OCH₃.

22. The method according to any of claims 1-18, wherein R₂ is CH₂-cyclopropyl.

23. The method according to any of claims 1-18, wherein R₄ and R₅ together form an oxo.

24. The method according to any of claims 1-18, wherein R₆ is CH₃.

25. The method according to any of claims 1-24, further comprising reacting a compound having the formula: 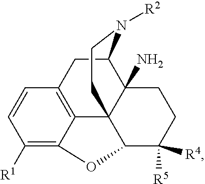
or a salt thereof,
with a compound having the formula: 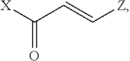
wherein X is halo, C₁₋₆alkoxy, C₆₋₁₀aryloxy, or C₁₋₆heteroaryl, each optionally substituted one or more times by a substituent in each case independently selected from halo, nitro, cyano, C₁₋₆alkyl, or C₁₋₆alkoxy;
and Z is optionally substituted aryl or heterocyclyl,
to give a compound having the formula: 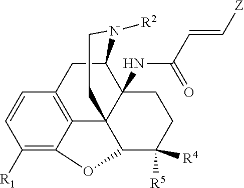
or a salt thereof.

26. The method according to claim 25, wherein Z has the formula: 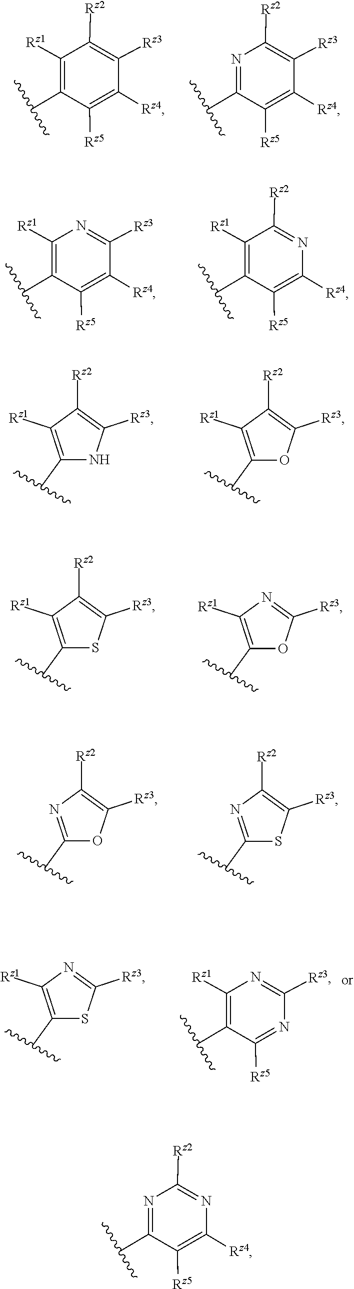
wherein:
R^{z1} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z2} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z3} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z4} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
R^{z5} is H, D, OH, NH₂, F, Cl, Br, I, COOH, C(═)OOC₁₋₄alkyl, C₁₋₄alkyl, OC₁₋₄alkyl, NHC₁₋₄alkyl, N(C₁₋₄alkyl)₂; wherein each C₁₋₄alkyl is optionally substituted one or more times by D, halo, OH, COOH, or NH₂;
wherein any two adjacent R^{z} groups may together form a fused aryl or heterocyclyl ring.

27. The method according to any of claims 1-24, further comprising reacting a compound having the formula: 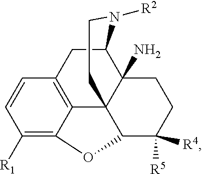
or a salt thereof,
with a compound having the formula: 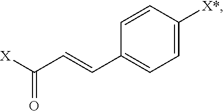
wherein X is halo, C₁₋₆alkoxy, C₆₋₁₀aryloxy, or C₁₋₆heteroaryl, each optionally substituted one or more times by a substituent in each case independently selected from halo, nitro, cyano, C₁₋₆alkyl, or C₁₋₆alkoxy;
and X* is H, C₁₋₄alkyl, halo, or nitro,
to give a compound of Formula (3): 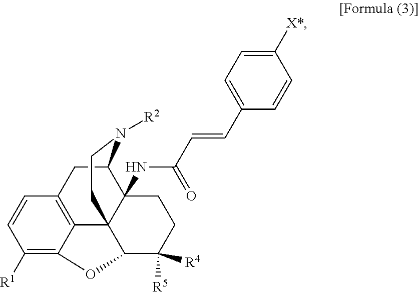
or a salt thereof.

28. The method according to claim 27, wherein X* is CH₃.

29. The method according to claim 27, wherein R* has the formula:

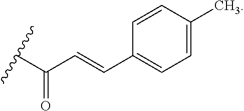 [SMILES: *C(=O)/C=C/c1ccc(C)cc1]

30. The method according to any of claims 1-29, further comprising reacting a compound having the formula: 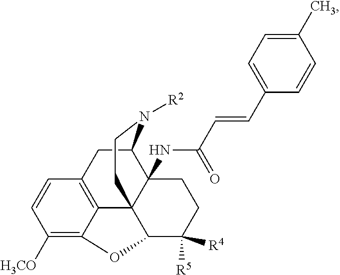
or a salt thereof,
with a demethylating agent to obtain a compound of Formula (4): 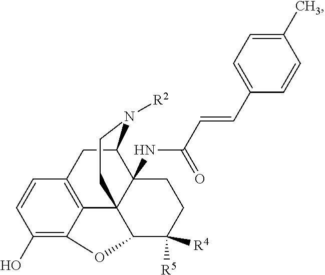
or a salt thereof.

31. The method according to claim 30, wherein the demethylating agent comprises BBr₃, LiPPH₂, L-selectride, HBr, 2-(diethylamino)ethanethiol, or a combination thereof.

32. MCAM free base Form 1.

33. MCAM maleate salt Form 2.

34. The MCAM maleate salt according to claim 33, characterized by x-ray diffraction peaks (°2Θ) of 7.9±0.2, 13.7±0.2, and 16.3±0.2.

35. The MCAM maleate salt according to claim 33 or 34, characterized by x-ray diffraction peaks (°2Θ) of 9.8±0.2, 10.2±0.2, 15.1±0.2, 19.4±0.2, 21.4±0.2, and 22.8±0.2.

36. The MCAM maleate salt according to any of claims 33-35, characterized by x-ray diffraction peaks (°2Θ) of 11.8±0.2, 12.7±0.2, 14.2±0.2, 21.7±0.2, and 24.5±0.2.

37. The MCAM maleate salt according to any of claims 33-36, characterized by five or more x-ray diffraction peaks (°2Θ) selected from 14.5±0.2, 16.8±0.2, 17.4±0.2, 18.1±0.2, 18.6±0.2, 20.2±0.2, 22.0±0.2, 22.4±0.2, 23.5±0.2, 24.9±0.2, 25.4±0.2, and 26.4±0.2.
